(19) United States
(12) Patent Application Publication
(10) Pub. No.: US 2007/0009584 A1
(43) Pub. Date: Jan. 11, 2007

(54) Manually-operable wound pressurization/depressurization system and method

(76) Inventor: Syde A. Taheri, Williamsville, NY (US)

Correspondence Address: WALTER W. DUFT;LAW OFFICES OF WALTER W. DUFT, 8616 MAIN ST, SUITE 2, WILLIAMSVILLE, NY, 14221, US

(21) Appl. No.: 11/360,850

(22) Filed: Feb. 23, 2006

Related U.S. Application Data
(60) Provisional application No. 60/698,034, filed on Jul. 11, 2005.
(60) Provisional application No. 60/726,072, filed on Oct. 11, 2005.

(51) Int. Cl. A61F 5/00; A61L 15/00

(52) U.S. Cl. 424/445; 602/1

(57) ABSTRACT

A wound treatment apparatus may include an impermeable or semi-permeable dressing, a securement strap on the dressing for securing the dressing over a wound on a patient, and a pressure/vacuum generator in fluid communication with the dressing. A wound treatment method utilizing the wound treatment apparatus may include placing the dressing of the apparatus over a wound, securing the securement strap of the apparatus around a body portion of a patient where the wound is situated, and manually operating the pressure/vacuum generator to selectively deliver positive or negative air pressure to the dressing and the wound. In another aspect directed to leg wound care, a wound treatment apparatus may include an impermeable or semi-permeable dressing and a vacuum generating foot pump in fluid communication with the dressing. The foot pump is manually operable by a patient and adapted to deliver air pressure (negative or positive) to the dressing and the wound.

### CROSS-REFERENCE TO RELATED APPLICATIONS

This application claims the benefit of U.S. Provisional Application Nos. 60/698,034 filed on Jul. 11, 2005 and 60/726,072 filed on Oct. 11, 2005.

### STATEMENT REGARDING FEDERALLY SPONSORED RESEARCH OR DEVELOPMENT

Not Applicable

BACKGROUND OF THE INVENTION

1. Field of the Invention

This invention relates to the healing of wounds, and particularly to wound dressings and related apparatus that deliver therapeutic treatment to a wound site.

2. Description of the Prior Art

By way of background, certain types of wounds require advanced healing therapies that cannot be provided by ordinary (passive) wound dressings. Examples include chronic open wounds, diabetic ulcers, venous stasis and vasculitic ulcers, acute and traumatic wounds, flaps and grafts, dehisced wounds and partial thickness burns. Vacuum-assisted closure (VAC) is one type of advanced wound healing therapy that has been used to manage and treat difficult wounds. According to this technique, a dressing having an impermeable or semi-permeable membrane is secured over a wound site and a vacuum source is connected to the dressing by way of a tube. The vacuum source operates continuously or periodically to apply negative air pressure to the wound site. The negative air pressure helps promote wound healing by removing infectious material and other fluids from the wound site. The dressing can also be connected to a source of liquid medication that can be delivered to the wound site during periods when no vacuum is applied.

It is to improvements in the healing of wounds and to wound treatment apparatus that the present invention is directed. In particular, what is needed is a new approach that targets wound treatment of the outer extremities, particularly the legs, and which speeds up the wound healing process, reduces edema and acts to prevent deep venous thrombosis.

### SUMMARY OF THE INVENTION

An advance in the art of wound treatment is provided by a wound treatment apparatus and related wound treatment method. The wound treatment apparatus may include an impermeable or semi-permeable dressing, a securement strap on the dressing for securing the dressing over a wound on a patient, and a pressure/vacuum generator in fluid communication with the dressing. The pressure/vacuum generator is adapted to periodically deliver positive air pressure and negative air pressure to the dressing and the wound. The securement strap is adapted to stabilize the dressing over the wound without substantial loss of air pressure when the pressure/vacuum generator delivers positive air pressure to the wound.

According to exemplary illustrated embodiments, the dressing may include a suction cup and can be provided with a connection fitting for releasably connecting the dressing to a mating connection fitting on a pressure/vacuum delivery tube that extends to the pressure/vacuum generator. The securement strap may include a releasable fastener or it may be formed as an uninterrupted elastic member. The securement strap could also be adjustable and may be sized to encircle an arm or a leg. The pressure/vacuum generator may be adapted for manual operation by the patient. For example, it could be implemented as a hand or foot pump. The wound treatment apparatus may also include a switch for switching between a positive air pressure mode and a negative air pressure mode. The apparatus may further include a pressure gauge to measure air pressure delivered by the pressure/vacuum generator, and a collection container for collecting fluid removed from the wound.

The wound treatment method is a method for using the wound treatment apparatus. The method may include placing the dressing of the apparatus over a wound, securing the securement strap of the apparatus around a body portion of a patient where the wound is situated, and manually operating the pressure/vacuum generator to selectively deliver positive or negative air pressure to the dressing and the wound. According to exemplary illustrated embodiments, the dressing may be placed on an arm or leg wound of the patient, the securement strap may be wrapped around the arm or leg that has the wound, and the pressure/vacuum generator may be a hand or foot pump operated using the wounded arm or leg. The dressing may include comprise a connection fitting for releasably connecting the dressing to a mating connection fitting on a vacuum delivery tube that extends to the foot pump. The method may then further include connecting the mating connection fitting to the connection fitting after placing the dressing over the wound. The method may additionally include disconnecting the mating connection fitting from the connection fitting when the fluid collection vessel has filled with fluid, then disposing of the foot pump and the fluid collection vessel.

In another aspect of the invention directed to leg wound care, the wound treatment apparatus may include an impermeable or semi-permeable dressing and a vacuum generating foot pump in fluid communication with the dressing. The foot pump is manually operable by a patient's foot and adapted to deliver air pressure (negative or positive) to the dressing and the wound. According to exemplary illustrated embodiments, the dressing may include a suction cup and a connection fitting for releasably connecting the dressing to a mating connection fitting on a vacuum delivery tube that extends to the foot pump. The apparatus may further include a pressure gauge to measure air pressure delivered by the foot pump. The apparatus may also include a collection container for collecting fluid removed from the wound.

A wound treatment method for leg wounds uses the leg wound treatment apparatus. The method includes placing the dressing over a leg wound, and manually pumping the foot pump using the leg having the wound to deliver air pressure (negative or positive) to the dressing and the leg wound. According to exemplary illustrated embodiments the dressing may comprises a connection fitting for releasably connecting the dressing to a mating connection fitting on a vacuum delivery tube that extends to the foot pump. The method may then further include connecting the mating connection fitting to the connection fitting after placing the dressing over the wound. The method may additionally include disconnecting the mating connection fitting from the connection fitting when the fluid collection vessel has filled with fluid, then disposing of the foot pump and the fluid collection vessel.

### BRIEF DESCRIPTION OF THE DRAWINGS

The foregoing and other features and advantages of the invention will be apparent from the following more particular description of preferred embodiments of the invention, as illustrated in the accompanying Drawings, in which:

FIG. 1 is schematic illustration of a wound treatment apparatus constructed in accordance with one aspect of the present invention;

FIG. 2 is schematic illustration of a wound treatment apparatus constructed in accordance with another aspect of the invention;

FIG. 3 is schematic illustration of a wound treatment apparatus constructed in accordance with a further aspect of the invention; and

FIG. 3 is schematic illustration of a wound treatment apparatus constructed in accordance with a still further aspect of the invention;

### DETAILED DESCRIPTION OF EXEMPLARY EMBODIMENTS

Turning to the drawings wherein like reference numerals indicate like elements in all of the several views, improved apparatus and methods for the treatment of wounds will now be described. These apparatus and methods are based on the recognition of two treatment modalities (useable separately or together) as rational alternatives to the conventional vacuum assisted closure method described by way of background above. The first treatment modality involves the intermittent application of negative and positive air pressure to a wound, with the negative pressure cycle being used to draw unwanted fluids away from the wound site, and the positive pressure cycle being used to direct air to the wound site to promote drying and healing. The second treatment modality involves the manual operation of a pumping apparatus by the patient to deliver positive and/or negative pressure to a wound site. The pumping apparatus is preferably operated using muscles that are proximate to the wound site. For example, if the wound is on a patient's leg, the pumping apparatus may be operated by flexing the wounded leg. If the wound is on a patient's arm, the pumping apparatus may be operated by flexing the wounded arm. If the wound is on a patient's torso, the pumping apparatus may be operated by moving the torso. In each of the foregoing examples, wound healing will be promoted as a result of increased blood flow to the wound site due to the flexing of adjacent muscle groups. This technique also promotes the reduction of edema and deep venous thrombosis by counteracting fluid retention and blood stagnation. The technique is also very suitable for home care and mobile applications insofar as no electrical power is required. The wound treatment apparatus can also be made at relatively low cost, could even be disposable.

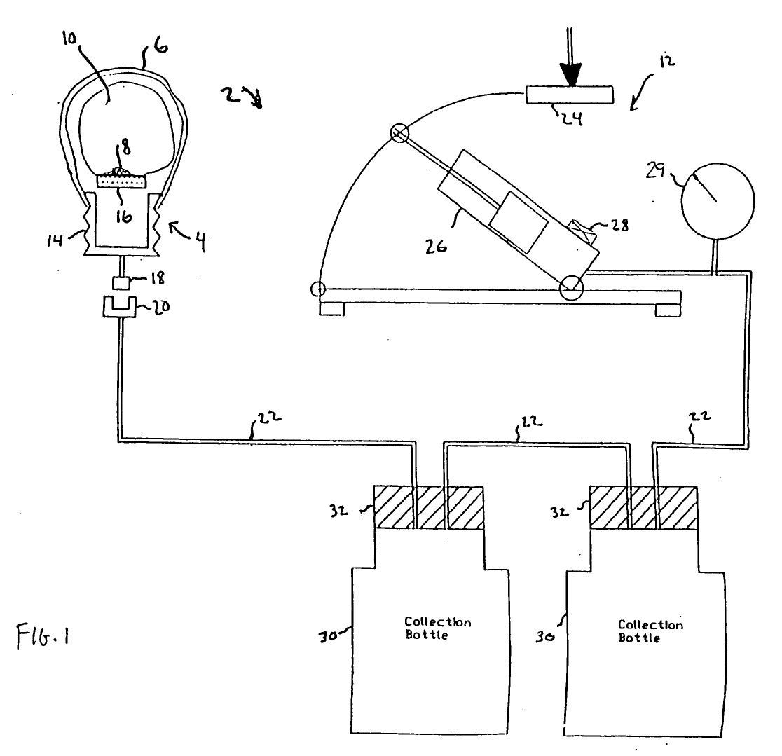

Turning now to FIG. 1, a wound treatment apparatus 2 includes an impermeable or semi-permeable dressing 4, a securement strap 6 on the dressing for securing the dressing over a wound 8 on a leg 10 (or other body portion) of a patient, and a pressure/vacuum generator 12 in fluid communication with the dressing. The pressure/vacuum generator 12 is adapted to periodically deliver positive air pressure and negative air pressure to the dressing 4 and the wound 8 that it covers. The securement strap 6 is adapted to stabilize the dressing 4 over the wound 8 without substantial loss of air pressure when the pressure/vacuum generator 12 delivers positive air pressure to the wound.

In the illustrated embodiment of FIG. 1, the dressing 4 includes a suction cup 14 made of impermeable or semi-impermeable material (such as rubber or plastic) mounted over a conventional absorbent dressing pad 16. The dressing 4 is further provided with a connection fitting 18 that extends from the suction cup 14 for releasably connecting the dressing to a mating connection fitting 20 on a pressure/vacuum delivery tube system 22 that extends to the pressure/vacuum generator 12. The securement strap 6 is shown to be formed as an uninterrupted elastic member, but could also be implemented as a two piece strap with a releasable fastener (e.g., a buckle, hook and loop connectors, etc.) for joining the two strap pieces. In either case, the securement strap will be adjustable, such that it may sized to encircle the leg 10 (or other body portion).

The pressure/vacuum generator 12 is adapted for manual operation by the patient. In particular, it is implemented as a foot pump having a foot pedal 24 operatively connected to a pneumatic pumping unit 26. The pressure/vacuum generator 12 also includes a switch 28 on the pumping unit 26 for switching between a positive air pressure mode and a negative air pressure mode. An optional pressure gauge 29 may be provided to measure negative air pressure delivered by the pressure/vacuum generator 12.

The wound treatment apparatus 2 is completed by at least one collection container 30 (two are shown) for collecting fluid removed from the wound 8 during the vacuum mode of operation. The collection containers 30 have pressure sealable closures 32 that interconnect the tubes of the tube system 22 in a continuous pressure/vacuum circuit that extends between the dressing 4 and the pressure/vacuum generator 12. Although the collection containers 30 are shown in FIG. 1 as being bottles, they could also be disposable bags or any other suitable collection vessel.

The wound treatment apparatus 2 may be used according to a wound treatment method that begins with optional irrigation and/or debridment of the wound 8. Advantageously, this can be done by operating the wound treatment apparatus 2 to deliver sterile saline or other irrigation solution under pressure to the wound site. In that case, a bag or other container containing the irrigation solution would be connected to the tube system 22 (or the collection container(s) 30 could be used to hold irrigation fluid) and the dressing pad 16 would be removed from the suction cup 14. After securing the suction cup 14 to the patient's leg (or other body portion) using the securement strap 6, the wound treatment apparatus 2 would be operated in the positive pressure mode for a desired period of time to deliver the irrigation solution under pressure to the wound 8.

Following the optional irrigation and/or debridment, the wound treatment method procedure entails (1) placement of the dressing 4 over the wound 8, (2) securement of the securement strap 6 around the patient's leg 10 (or other body portion), (3) interconnection of the connection fittings 18 and 20 (as necessary), and (4) manual operation of the pressure/vacuum generator 12 via dorsi flexion of a foot to generate positive or negative air pressure. Assuming the wound 8 is a leg wound, the foot that operates the pressure/vacuum generator 12 will preferably be part of the leg 10 so that muscles proximate to the wound 8 will be flexed during treatment. The duration of treatment will be dictated by medical and therapeutic requirements. By way of example only, a treatment session of 5-10 minutes per hour could be used.

The switch 28 on the pumping unit 26 is utilized to periodically switch between the positive and negative pressure modes. The time spent in each treatment mode may vary according patient needs. For example, if therapy is delivered for 10 minutes every hour, there could be 5 minutes of negative pressure followed by 5 minutes of positive pressure, 8 minutes of negative pressure followed by 2 minutes of positive pressure, 6 minutes of positive pressure followed by 4 minutes of negative pressure, and so on.

In the negative pressure mode, the pumping unit 26 is manually operated by the patient pressing up and down on the foot pad 24 to deliver negative air pressure to the dressing 4 and the wound 8. The negative pressure generated by the pressure/vacuum generator 12 will be applied through the tube system 22 to the dressing 4, and thus to the wound 8. Unwanted fluids will be drawn out of the wound 8 and deposited to the collection containers 30, which can be periodically emptied. The connection fitting 20 can be detached from the connection fitting 18 to facilitate removal of the collection containers 30 from the vicinity of the patient. Depending on the construction of the pressure/vacuum generator 12, the tubing 22 and the collection containers 30, these items could simply be disposed of instead of emptying fluid from the collection containers and reusing them. Alternatively, just the collection containers 30 and the tubing 22 could be disposed of, while leaving the pressure/vacuum generator 12 for re-use.

In the positive pressure mode, the foot that operates the wound treatment apparatus 2 will again press up and down on the foot pad 24 and pressurized air will be delivered through the tubing 22 from the pressure/vacuum generator 12 to the dressing 4 and the wound 8. The application of positive air pressure will promote drying and healing of the wound 8.

As described above, if the patient operates the pressure/vacuum generator 12 using his/her leg 10, wound healing will be promoted by increasing blood flow to the wound site due to the flexing of adjacent muscle groups. This technique should also promote the reduction of edema and deep venous thrombosis as a result of increased blood flow.

According to an alternative wound treatment method, the wound treatment apparatus 2 could be used in the negative pressure (vacuum) mode only. In that case, the securement strap 6 may not be needed because the negative pressure would tend to hold the suction cup 14 against the patient's leg 10 (or other body portion). Alternatively, adhesive could be used to minimally secure the suction cup 14 during periods when negative pressure is not being applied. According to another alternative leg wound treatment method, the wound treatment apparatus 2 could be used in a positive pressure mode only. In that case, the securement strap 6 will be used.

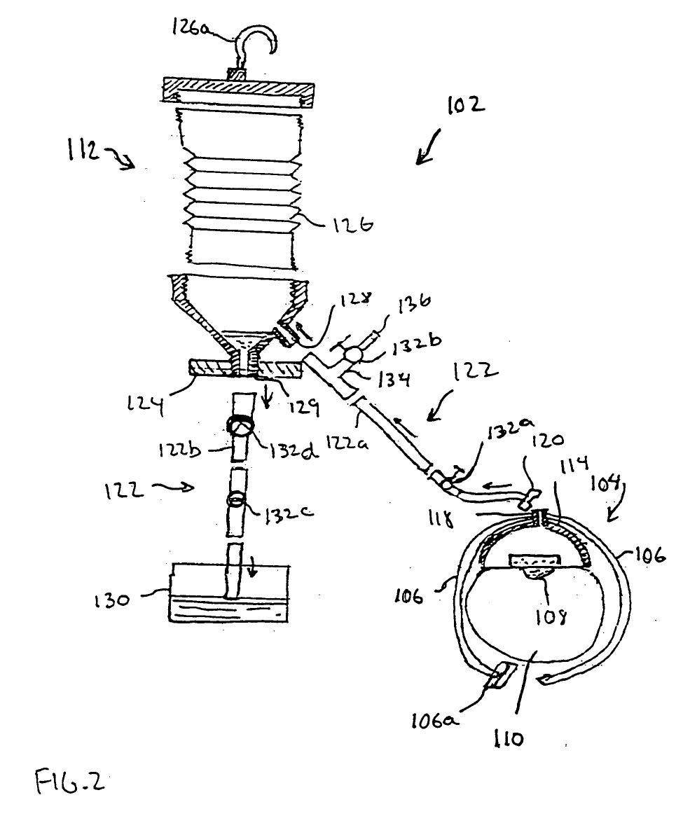

Turning now to FIG. 2, an alternative wound treatment apparatus 102 is shown that includes an impermeable or semi-permeable dressing 104, a securement strap 106 on the dressing for securing the dressing over a wound 108 on an arm 110 (or other body portion) of a patient, and a pressure/vacuum generator 112 in fluid communication with the dressing. The pressure/vacuum generator 112 is adapted to periodically deliver positive air pressure and negative air pressure to the dressing 104 and the wound 108 that it covers. The securement strap 106 is adapted to stabilize the dressing 104 over the wound 108 without substantial loss of air pressure when the pressure/vacuum generator 112 delivers positive air pressure to the wound.

In the illustrated embodiment of FIG. 2, the dressing 104 includes a suction cup 114 made of impermeable or semi-impermeable material (such as rubber or plastic) mounted over a conventional absorbent dressing pad 116. The dressing 104 is further provided with a connection fitting 118 that extends from the suction cup 114 for releasably connecting the dressing to a mating connection fitting 120 on a pressure/vacuum delivery tube system 122 that extends to the pressure/vacuum generator 112 (and thereafter to a collection container). The securement strap 106 is shown to be formed as a two piece strap with a releasable buckle fastener 106a (other connectors could also be used) for joining the two strap pieces. In an alternative construction (not shown), the securement strap 106 could be an uninterrupted resilient member. In either case, the securement strap will be adjustable, such that it may sized to encircle the arm 110 (or other body portion).

The pressure/vacuum generator 112 is adapted for manual operation by the patient. In particular, it is implemented as a hand pump having a handle 124 operatively connected to a pneumatic bellows pumping unit 126. The pumping unit 126 includes a first port 128 that connects to a first portion 122a of the tubing system 122, and a second port 129 that connects to a second portion 122b of the tubing system 122. A hook 126a (or other suitable connector) is located on the top of the pumping unit 126 for securing the pumping unit to a support structure (not shown).

The wound treatment apparatus 102 further includes a collection container 130 for collecting fluid removed from the wound 108 during the vacuum mode of operation. The collection container 130, which could be a bottle, bag or any other suitable vessel, is positioned to receive fluid dispensed from the free end of the tubing portion 122b. A series of valves are placed in the tubing portions 122a and 122b in order to control the positive and negative pressure cycles. The valves include a first valve 132a proximate to the dressing 104 in the tubing portion 122a, a second valve 132b proximate to the pressure/vacuum generator 112 in a tee-section 134 of the tubing portion 122a, a third valve 132c proximate to the collection container 130 in the tubing portion 122b, and a fourth valve 132d proximate to the pressure/vacuum generator 112 in the tubing portion 122b. The valves 132a, 132b and 132c are control valves (switches) that can be manually operated. The valve 132d is an optional check valve.

The wound treatment apparatus 102 may be used according to a wound treatment method that begins with optional irrigation and/or debridment of the wound 108 (e.g., by using the wound treatment apparatus 102 to deliver irrigation solution under pressure). This is followed by (1) placement of the dressing 104 over the wound 108, (2) securement of the securement strap 106 around the patient's arm 110 (or other body portion), (3) interconnection of the connection fittings 118 and 120 (as necessary), and (4) manual operation of the pressure/vacuum generator 112. This manual operation is achieved by gripping the handle 124 on the pressure/vacuum generator 112 and reciprocating the pumping unit 126 up and down to generate positive or negative air pressure. Assuming the wound 108 is an arm wound, the patient will preferably operate the pressure/vacuum generator 112 using his/her arm 110 so that muscles proximate to the wound 108 will be flexed during treatment. As described above, the duration of treatment will be dictated by medical and therapeutic requirements.

The control valves 132a, 132b and 132c are utilized to periodically switch between the positive and negative pressure modes. As described above, the time spent in each treatment mode will vary according patient needs. In the negative pressure mode, the valves 132a and 132c are closed, the valve 132b is opened, and the pumping unit 126 is contracted by pushing up on the handle 124. Air in the pumping unit 126 is expelled through the open port 136 in the tee-section 134. The valve 132b is then closed, the valve 132a is opened, and the pumping unit 126 is elongated by slowly pulling down on the handle 124. This introduces negative pressure in the tubing portion 122a and hence the dressing 104. Unwanted fluids will be drawn out of the wound 108 and passed through the tubing portion 122a to the pumping unit 126. After opening the valve 132c, the fluids will then drain through the port 129 and the tubing portion 122b, and deposit into the collection container 130, which can be periodically emptied. The foregoing process may be repeated for the next negative pressure cycle.

In the positive pressure mode, the valves 132a and 132c are closed and the valve 132b is opened. The pumping unit 126 is then elongated by pulling down on the handle 124. This draws air into the pumping unit 126 through an open port 136 in the tee-section 134. The valve 132b is then closed and the valve 132a is opened. The pumping unit 126 is then contracted by pushing upwardly on the handle 124. This expels the air in the pumping unit 126 into the tubing portion 122, and the air is delivered through the open valve 132a to the dressing 104 under positive pressure, and thus to the wound 108 to promote healing. The foregoing process may be repeated for the next positive pressure cycle.

As described above, if the wound 108 is on the arm 110 that operates the pressure/vacuum generator 112, wound healing will be promoted by increasing blood flow to the wound site due to the flexing of adjacent muscle groups. This technique should also promote the reduction of edema and deep venous thrombosis.

According to an alternative wound treatment method, the wound treatment apparatus 102 could be used in the negative pressure (vacuum) mode only. In that case, the securement strap 106 may not be needed because the negative pressure would tend to hold the suction cup 114 against the patient's arm 110 (or other body portion). Alternatively, adhesive could be used to minimally secure the suction cup 114 during periods when negative pressure is not being applied. According to another alternative wound treatment method, the wound treatment apparatus 102 could be used in a positive pressure mode only. In that case, the securement strap 106 will be used.

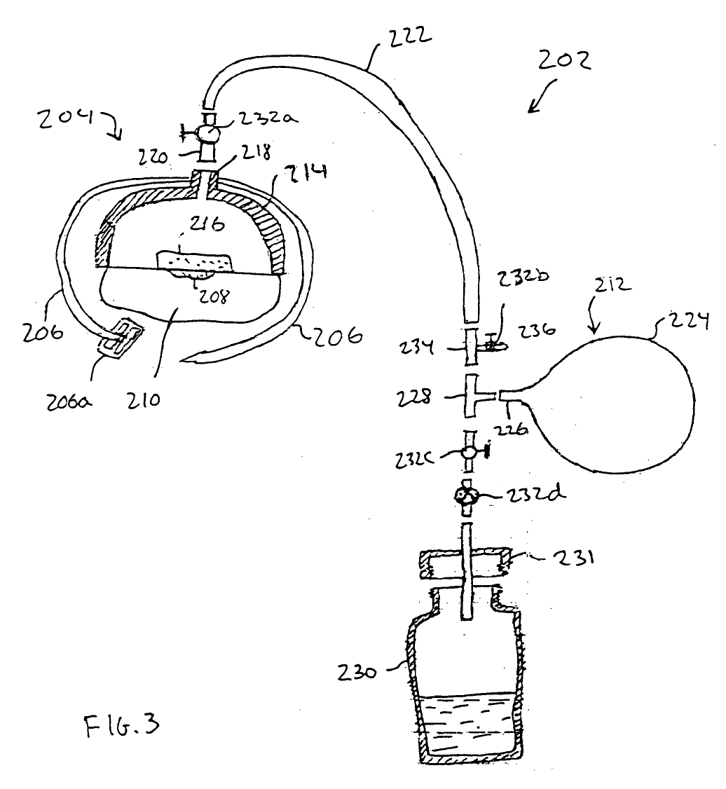

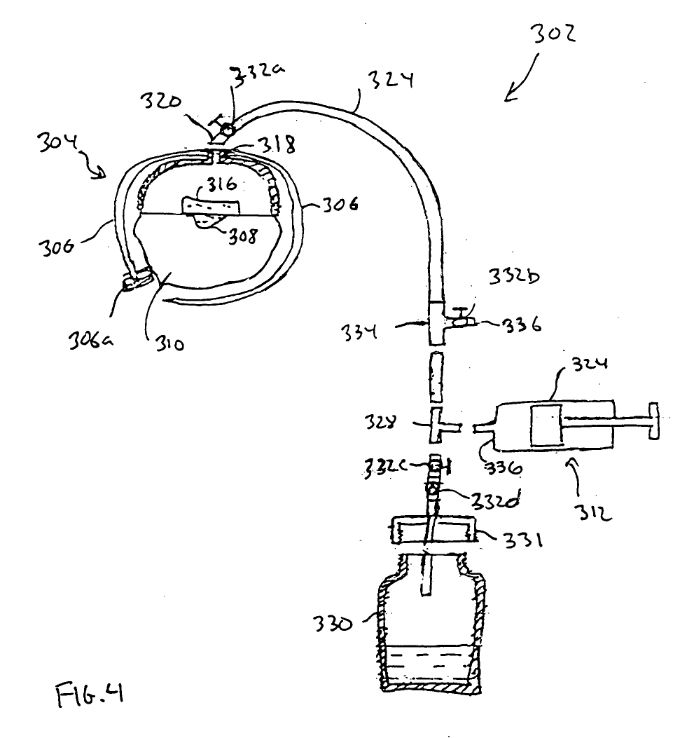

Turning now to FIG. 3, another alternative wound treatment wound treatment apparatus 202 is shown that includes an impermeable or semi-permeable dressing 204, a securement strap 206 on the dressing for securing the dressing over a wound 208 on an arm 210 (or other body portion) of a patient, and a pressure/vacuum generator 212 in fluid communication with the dressing. The pressure/vacuum generator 212 is adapted to periodically deliver positive air pressure and negative air pressure to the dressing 204 and the wound 208 that it covers. The securement strap 206 is adapted to stabilize the dressing 204 over the wound 208 without substantial loss of air pressure when the pressure/vacuum generator 212 delivers positive air pressure to the wound.

In the illustrated embodiment of FIG. 3, the dressing 204 includes a suction cup 214 made of impermeable or semi-impermeable material (such as rubber or plastic) mounted over a conventional absorbent dressing pad 216. The dressing 204 is further provided with a connection fitting 218 that extends from the suction cup 214 for releasably connecting the dressing to a mating connection fitting 220 on a pressure/vacuum delivery tube system 222 that extends to the pressure/vacuum generator 212. The securement strap 206 is shown to be formed as a two piece strap with a releasable buckle fastener 206a (other connectors could also be used) for joining the two strap pieces. In an alternative construction (not shown), the securement strap 106 could be an uninterrupted resilient member. In either case, the securement strap will be adjustable, such that it may sized to encircle the arm 210 (or other body portion).

The pressure/vacuum generator 212 is adapted for manual operation by the patient. In particular, it is implemented as a hand pump having an air bladder 224 that provides a pumping unit 226. The pumping unit is operatively connected to a tee-section 228 in the tubing 222.

The wound treatment apparatus 202 further includes a collection container 230 for collecting fluid removed from the wound 208 during the vacuum mode of operation. The collection container 230, which could be a bottle, a bag or any other suitable vessel, has a sealable closure 231 attached at the free end of the tubing 222 to receive fluid dispensed from the tubing. A series of valves are placed in the tubing 222 in order to control the positive and negative pressure cycles. The valves include a first valve 232a proximate to the dressing 204, a second valve 232b proximate to the pressure/vacuum generator 212 in a tee-section 234 of the tubing 222, a third valve 232c proximate to the collection container 230, and a fourth valve 232d also proximate to the collection container 230. The valves 232a, 232b and 232c are control valves (switches) that can be manually operated. The valve 232d is an optional check valve.

The wound treatment apparatus 202 may be used according to a wound treatment method that begins with optional irrigation and/or debridment of the wound 208 (e.g., by using the wound treatment apparatus 202 to deliver irrigation solution under pressure). This is followed by (1) placement of the dressing 204 over the wound 208, (2) securement of the securement strap 206 around the patient's arm 210 (or other body portion), (3) interconnection of the connection fittings 218 and 220 (as necessary), and (4) manual operation of the pressure/vacuum generator 212. This manual operation is achieved by gripping the bladder 224 and squeezing and releasing it to generate positive or negative air pressure. Assuming the wound 208 is an arm wound, the patient will preferably operate the pressure/vacuum generator 212 using his/her arm 210 so that muscles proximate to the wound 208 will be flexed during treatment. As described above, the duration of treatment will be dictated by medical and therapeutic requirements.

The control valves 232a, 232b and 232c are utilized to periodically switch between the positive and negative pressure modes. As described above, the time spent in each treatment mode will vary according patient needs. In the negative pressure mode, the valves 232a and 232c are closed, the valve 232b is opened, and the pumping unit 226 is contracted by squeezing the bladder 224. Air in the pumping unit 226 is expelled through the open port 236 in the tee-section 234. The valve 232b is then closed, the valve 232a is opened, and the pumping unit 226 is expanded by slowly releasing the bladder 224. This introduces negative pressure in the tubing 222 and hence the dressing 204. Unwanted fluids will be drawn out of the wound 208 and passed through the tubing 224 to the pumping unit 226. The valve 232a is then closed and the valve 232c is opened. The bladder 224 is again squeezed to expel the fluid therein through the tubing 222 to the collection container 230, which can be periodically emptied. The foregoing process may be repeated for the next negative pressure cycle.

In the positive pressure mode, the valves 232b and 232c are closed and the valve 232a is opened. The pumping unit 226 is then compressed by slowly squeezing the bladder 224. This forces air from the pumping unit 226 through the tubing 222 to the dressing 204 under positive pressure, and thus to the wound 208 to promote healing. The valve 232a is then closed, the valve 232b is opened, and the bladder 224 is released so that in will refill with air introduced through the port 236. The foregoing process may be repeated for the next positive pressure cycle.

As described above, if the wound 208 is on the arm 210 that operates the pressure/vacuum generator 212, wound healing will be promoted by increasing blood flow to the wound site due to the flexing of adjacent muscle groups. This technique should also promote the reduction of edema and deep venous thrombosis.

According to an alternative wound treatment method, the wound treatment apparatus 202 could be used in the negative pressure (vacuum) mode only. In that case, the securement strap 206 may not be needed because the negative pressure would tend to hold the suction cup 214 against the patient's arm 210 (or other body portion). Alternatively, adhesive could be used to minimally secure the suction cup 214 during periods when negative pressure is not being applied. According to another alternative arm wound treatment method, the arm wound treatment apparatus 202 could be used in a positive pressure mode only. In that case, the securement strap 206 will be used.

Turning now to FIG. 4, another alternative wound treatment apparatus 302 is shown that is substantially identical to the wound treatment apparatus 202 of FIG. 3, as shown by the use of corresponding reference numerals incremented by 100. The only difference is that the bladder 224 is replaced with a syringe 324. Thus, instead of squeezing and releasing a bladder, a syringe plunger is slid in and out to create positive and negative pressure. Except for the substitution of the syringe 324 for the bladder 224, the above discussion of the wound treatment apparatus 202 of FIG. 3 applies to the wound treatment apparatus 302 of FIG. 4, and will not be repeated.

Although not shown, the design principals of the wound treatment apparatus of FIGS. 1-4 could be used to create a wound treatment apparatus having a pressure/vacuum generator that can be activated by movement of a patient's torso. Such movements could include raising the torso from a prone position to a sitting position, rocking the torso while in a sitting position, or turning/twisting the torso while in a prone or sitting position. This design would be useful for treating wounds of the torso, in which case the torso movement required to operate the pressure/vacuum generator would produce beneficial blood flow to the wound.

Accordingly, various wound treatment apparatus have been disclosed in conjunction with related wound treatment methods. It will be appreciated that the objects of the invention have been achieved. The disclosed wound treatment apparatus allow for a combination of positive and negative pressure therapies, or either therapy alone. By providing for manual operation using muscles proximate to a wound site, edema and deep venous thrombosis can be reduced by counteracting fluid retention and blood stagnation. The approach is also very suitable for home care and mobile applications because no electrical power is required. The disclosed wound treatment apparatus may also be constructed at low cost, and could even be disposable. While various embodiments of the invention have been shown and described, it should be apparent that many variations and alternative embodiments could be implemented in accordance with the teachings herein. It is understood, therefore, that the invention is not to be in any way limited except in accordance with the spirit of the appended claims and their equivalents.

What is claimed is:

1. A wound treatment apparatus, comprising:
an impermeable or semi-permeable dressing;
a securement strap on said dressing for securing said dressing over a wound on a patient;
a pressure/vacuum generator in fluid communication with said dressing;
said pressure/vacuum generator being adapted to periodically deliver positive air pressure and negative air pressure to said dressing and said wound; and
said securement strap being adapted to stabilize said dressing over said wound without substantial loss of pressure when said pressure/vacuum generator delivers positive air pressure to said wound.

2. An apparatus in accordance with claim 1 wherein said dressing comprises a suction cup.

3. An apparatus in accordance with claim 1 wherein said dressing comprises a connection fitting for releasably connecting said dressing to a mating connection fitting on a pressure/vacuum delivery tube that extends to said pressure/vacuum generator.

4. An apparatus in accordance with claim 1 wherein securement strap comprises a releasable fastener.

5. An apparatus in accordance with claim 1 wherein said securement strap comprises an uninterrupted elastic member.

6. An apparatus in accordance with claim 1 wherein said securement strap is adjustable.

7. An apparatus in accordance with claim 1 wherein said securement strap is sized to encircle an arm or a leg.

8. An apparatus in accordance with claim 1 wherein said pressure/vacuum generator is adapted for manual operation by said patient.

9. An apparatus in accordance with claim 9 wherein said pressure/vacuum generator comprises a hand or foot pump.

10. An apparatus in accordance with claim 1 further including a switch for switching between a positive air pressure mode and a negative air pressure mode.

11. A method for using a wound treatment apparatus, comprising:
selecting a wound treatment apparatus having:
an impermeable or semi-permeable dressing;
a securement strap on said dressing for securing said dressing over a wound on a patient;
a pressure/vacuum generator in fluid communication with said dressing;
said pressure/vacuum generator being adapted to periodically deliver positive air pressure and negative air pressure to said dressing and said wound; and
said securement strap being adapted to stabilize said dressing over said wound without substantial loss of air pressure when said pressure/vacuum generator delivers positive air pressure to said wound;
placing said dressing over said wound;
securing said securement strap around a body portion of said patient where said wound is situated; and
manually operating said pressure/vacuum generator to selectively deliver positive or negative air pressure to said dressing and said wound.

12. A method in accordance with claim 11 wherein said dressing is placed on an arm or leg wound of said patient, said securement strap is wrapped around the arm or leg that has said wound, said pressure/vacuum generator is a hand or foot pump operated using said wounded arm or leg, and said hand or foot pump is operated to alternatingly deliver positive and negative air pressure to said dressing and said wound.

13. A wound treatment apparatus, comprising:
an impermeable or semi-permeable dressing;
a vacuum generating foot pump in fluid communication with said dressing; and
said foot pump being manually operable by a patient and adapted to deliver air pressure (negative or positive) to said dressing and said wound.

14. An apparatus in accordance with claim 13 wherein said dressing comprises a suction cup.

15. An apparatus in accordance with claim 13 wherein said dressing comprises a connection fitting for releasably connecting said dressing to a mating connection fitting on a vacuum delivery tube that extends to said foot pump.

16. An apparatus in accordance with claim 13 further including a pressure gauge to measure air pressure delivered by said foot pump.

17. An apparatus in accordance with claim 13 further including a collection container for collecting fluid removed from said wound.

18. A method for using a wound treatment apparatus to treat a leg wound, comprising:
selecting a wound treatment apparatus having:
an impermeable or semi-permeable dressing;
a collection container for collecting fluid removed from said wound;
a vacuum generating foot pump in fluid communication with said dressing; and
said foot pump being manually operable by a patient and adapted to deliver air pressure to said dressing and said leg wound;
placing said dressing over said leg wound; and
manually pumping said foot pump using the leg having said wound to deliver air pressure (negative or positive) to said dressing and said leg wound.

19. A method in accordance with claim 18 wherein said dressing comprises a connection fitting for releasably connecting said dressing to a mating connection fitting on a vacuum delivery tube that extends to said foot pump, and wherein said method further includes connecting said mating connection fitting to said connection fitting prior to or after placing said dressing over said wound.

20. A method in accordance with claim 19 further including disconnecting said mating connection fitting from said connection fitting when said fluid collection vessel has filled with fluid, then disposing of said foot pump and said fluid collection vessel.
